(19) United States
(12) Patent Application Publication
(10) Pub. No.: US 2011/0190734 A1
(43) Pub. Date: Aug. 4, 2011

(54) Mechanical Advantage For Hub Linear Travel For A Drainage Catheter

(76) Inventors: Richard Graffam, Marlborough, MA (US); Ben Morris, Jeffersonville, IN (US); Chuck Bourgeois, Wilmington, MA (US)

(21) Appl. No.: 12/699,714

(22) Filed: Feb. 3, 2010

(51) Int. Cl. A61M 27/00

(52) U.S. Cl. 604/540

(57) ABSTRACT

An indwelling drainage catheter is disclosed that is configured to include a longitudinal catheter shaft having a distal and a proximal end, a cord in coaxial arrangement within said longitudinal catheter, a static hub operatively connected to said longitudinal catheter at said proximal end, a dynamic hub configured to move along a longitudinal axis of said longitudinal catheter substantially near said proximal end, wherein a first end of said cord is affixed to said longitudinal catheter at said proximal end and a second end of said cord is affixed to said longitudinal catheter at said distal end, and further wherein the longitudinal movement of the dynamic hub is substantially one half the distance needed to close the pigtail shape.

### FIELD

The present invention relates generally to indwelling drainage catheters, and more particularly, to a catheter that is configured to form a pigtail curve upon the relatively short longitudinal manipulation of a dynamic hub, which allows the catheter to be introduced and locked into a body cavity.

### BACKGROUND

Flexible catheters are used for percutaneous drainage of an abscess or pocket of fluid in the body to the exterior by means of gravity or negative pressure. Fluid collection may be the result of an infection, surgery, trauma or other causes. Typical fluids include biliary, nephrostomy, pleural, urinary, and mediastinal collections. As an alternative to providing drainage, these catheters can also be used to introduce substances, such as fluids, into a patient's body.

In percutaneous drainage procedures, a catheter is typically introduced into a patient through a hypodermic needle or a trocar. A guidewire is inserted through the needle, which is then removed. The catheter tube, with a stiffening cannula, then passes over the previously emplaced guide wire into the drainage site in the body cavity. The stiffening cannula is then removed.

Once a drainage catheter is in position in the body cavity, it is desirable to anchor the catheter before drainage begins. Typically, this can be done by forming a restraining portion in the distal end of the catheter in the form of a pigtail or “J-curve.” For a pigtail configuration, a flexible tension member, such as a suture thread, extends through draw ports at two spaced positions along the distal portion of the catheter. The restraining portion is conventionally activated by manually pulling the suture thread so that the two draw ports move toward each other as the pigtail loop forms at the distal end of the catheter. When the suture thread is taut, it prevents the pigtail loop from straightening by holding the juxtaposed portions of the catheter together in a locked position. The restraining portion is thus in a shape capable of resisting displacement from the body cavity. Once actuated, this restraining portion prevents removal of the catheter. When the catheter is ready to be removed, the cannula is inserted through the lumen until it reaches the pigtail loop. The restraining portion at the distal end is unlocked by cutting or releasing the suture at the proximal end, where the catheter protrudes from the body. Then the stiff cannula can be advanced distally to straighten the pigtail and help remove the catheter from the patient.

A preformed curve in the shape of a malecot rib has also been used as a possible anchoring mechanism. In this configuration, longitudinal slits are located in the restraining portion of the catheter at the distal end. The rib is activated in a similar manner as the pigtail configuration by manipulating a tension member, except the restraining portion is formed in the shape of multiple wings (typically two or four) instead of a pigtail.

Successful procedures involving percutaneous drainage depend upon the initial placement of the drainage catheter and having the catheter remain in place for the duration of the treatment. Without adequate anchoring or support, catheter dislodgment may result due to body movements by the patient or under other conditions.

Described herein are unique devices, systems and methods for supplementing the pigtail or malecot anchoring mechanism by using a catheter that is configured to form a pigtail curve upon the longitudinal manipulation of a dynamic hub.

### SUMMARY

The devices, systems and methods described herein relate to drainage catheters and an anchoring structure or mechanism for indwelling catheters (both short and long-term). This feature allows for the catheter to be introduced or locked into the anatomy via stabilized formation of a pigtail structure at the drainage catheter tip.

In one embodiment, the catheter includes a longitudinal catheter shaft having a distal and a proximal end, a cord in coaxial arrangement within said longitudinal catheter, a static hub operatively connected to said longitudinal catheter at said proximal end, a dynamic hub configured to move along a longitudinal axis of said longitudinal catheter substantially near said proximal end, wherein a first end of said cord is affixed to said longitudinal catheter at said proximal end and a second end of said cord is affixed to said longitudinal catheter at said distal end, and further wherein the longitudinal movement of the dynamic hub is substantially one half the distance needed to close the pigtail shape.

In another embodiment, the catheter includes a catheter means having a distal and a proximal end, a cord means in coaxial arrangement within said catheter, a first hub means operatively connected to said catheter means at said proximal end, a second hub means configured to move along a longitudinal axis of said catheter means, and wherein a first end of said cord means is affixed to said catheter means at said proximal end and a second end of said cord means is affixed to said catheter means at said distal end, and further wherein longitudinal movement of said second hub is substantially one half the distance needed to close the pigtail shape.

A method of affixing a catheter within a body cavity includes inserting a distal end of a catheter into a body cavity, the catheter comprising a longitudinal catheter shaft having a distal and a proximal end, a cord in coaxial arrangement within said longitudinal catheter, a static hub operatively connected to said longitudinal catheter at said proximal end, a dynamic hub configured to move along a longitudinal axis of said longitudinal catheter substantially near said proximal end and resting in a disengaged state wherein a first end of said cord is affixed to said dynamic hub and a second end of said cord is affixed to said longitudinal catheter at said distal end; and securing the catheter within the body cavity by engaging said dynamic hub to said static hub and causing the distal portion said longitudinal catheter to form a pigtail curve.

Of the various features described, the structures herein offer a number of advantages in their construction and ability to anchor the drainage catheter in various applications. Other systems, methods, features and advantages will be or will become apparent to one with skill in the art upon examination of the following figures and detailed description. It is intended that all such additional systems, methods, features and advantages be included within this description, be within the scope of the devices, systems and methods described herein, and be protected by the accompanying claims.

### BRIEF DESCRIPTIONS OF THE DRAWINGS

The figures provided herein are not necessarily drawn to scale, with some components and features being exaggerated for clarity. Each of the figures diagrammatically illustrates aspects of the embodiments.

FIG. 1 is a perspective view of a catheter with a “pig tail” loop configuration as an anchoring mechanism, shown after the activation of the pig tail.

FIG. 2 is a perspective view of a catheter with a “pig tail” loop configuration as an anchoring mechanism, shown before the activation of the pig tail.

FIGS. 3a, 3b, and 3c are cross-sectional views depicting an exemplary embodiment of a catheter with a “pig tail” loop configuration as an anchoring mechanism, with the dynamic hub in a completely disengaged position, partially disengaged position, and an engaged position, respectively.

FIG. 4 is a cross-sectional view of the proximal end of a catheter, specifically focused on the internal construction of the dynamic hub.

### DETAILED DESCRIPTION

The devices, systems and methods described herein can be used for introducing a percutaneous catheter into a patient and anchoring the catheter into the body of the patient to facilitate draining fluid or removing other materials from the body. Alternatively, the catheter can introduce substances, such as fluids, into the patient's body.

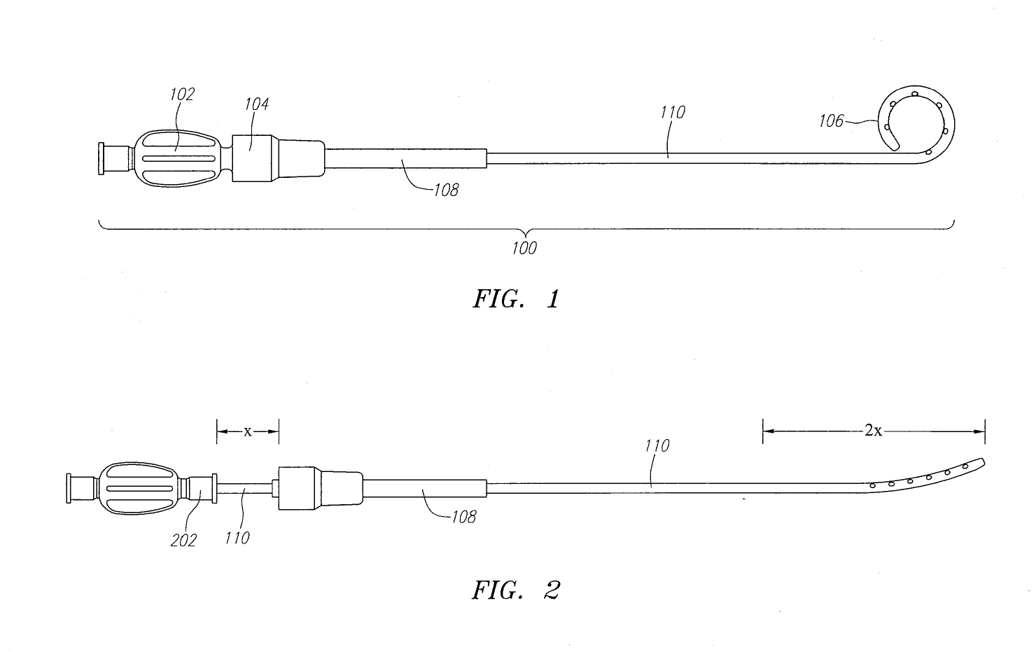

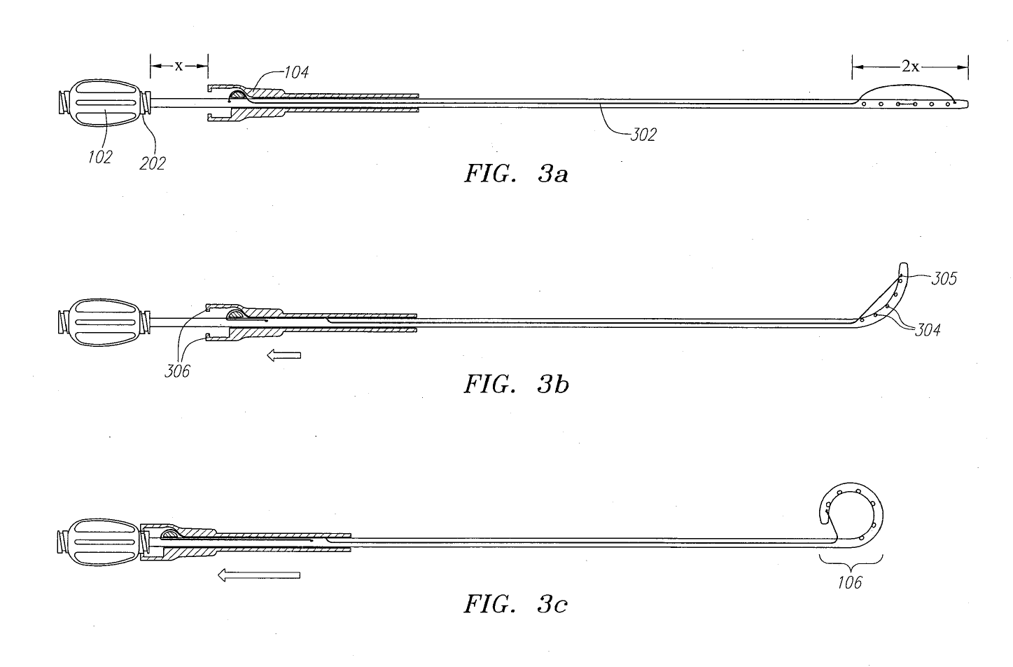

Referring to FIG. 1, a perspective view of a catheter 100 with a “pig tail” loop 106 configuration as an anchoring mechanism, shown after the activation of the pig tail 106, is shown. FIG. 1 depicts the distal portion of a catheter 100 comprising a flexible, elongate tube member 110 and a restraining portion, the restraining portion comprising the pigtail loop 106. The wall of the drainage catheter 100 toward the distal end includes a series of drainage holes, or perforations 304 (See FIG. 3). The pigtail loop 106 maintains its “pigtail” formation by manipulating the dynamic hub 104 along a longitudinal axis of the catheter 100. As the dynamic hub 104 is moved in a proximal direction towards the static hub 102, a cord 302 draws the distal end of the tube member 110 into a pigtail loop configuration (See FIG. 3). The dynamic hub 104 and the static hub 102 may be temporarily mated by an engaging member 202 and a corresponding mating member 306 of the dynamic hub 104. The engaging member 202 and corresponding mating member 306 may be an elementary threaded application. The mating feature of the dynamic hub 104 and the static hub 102 enables the pigtail loop 106 to maintain its shape since, in the fully engaged position, the cord 302 is longitudinally drawn in the proximal direction and affixes the distal end of the catheter 100 onto itself.

Once the dynamic hub 104 and the static hub 102 are disengaged, the dynamic hub 104 may move distally along the longitudinal axis of the catheter 100. As the dynamic hub 104 is manipulated in a distal direction, the cord 304 retracts and loosens, thus, the pigtail loop 106 has the ability to flatten and return to its substantially straightened position. Because the tube member 110 may have been in a pigtail formation for a length of time, it may be helpful, but not necessary, to insert a stylet to straighten the catheter lumen and facilitate extraction of the catheter 100 from the body cavity. Likewise, when the catheter 100 is first introduced into a patient (not shown), a cannula, stylet, or other rigid straightening device may be inserted into the lumen of the tube member 110 to help straighten the catheter 100 and facilitate insertion. When the distal tip of the catheter 100 reaches the drainage site, the stylet is proximally withdrawn, and the dynamic hub 104 is then moved towards the proximal static hub 102 so as to draw the drainage holes 304 closer together to form the pigtail loop 106 configuration. As a result, the pigtail loop 106 configuration is formed, as shown in FIG. 1.

Referring to FIG. 2, a perspective view of a catheter 100 with a “pig tail” loop 106 configuration as an anchoring mechanism is shown. FIG. 2 depicts the catheter 100 in a substantially straight configuration with the dynamic hub 104 and static hub 102 disengaged. The elongate tube member 110 defines an internal lumen, which extends through the catheter 100. Typically, when employing the catheter 100 for use into a body cavity, the elongate tube member 110 has a diameter of between 6 and 18 French (Fr). Moreover, as the dynamic hub 104 is moved distally along the elongate member 110, thus exposing said elongate member 110 as a distance X, the distal-most portion of the catheter 100 relaxes into a substantially straightened position that covers the distance 2×. In other words, for every fractional movement of the dynamic hub 104 along a longitudinal axis, the distal-most portion of the catheter 100 is manipulated by a factor of two.

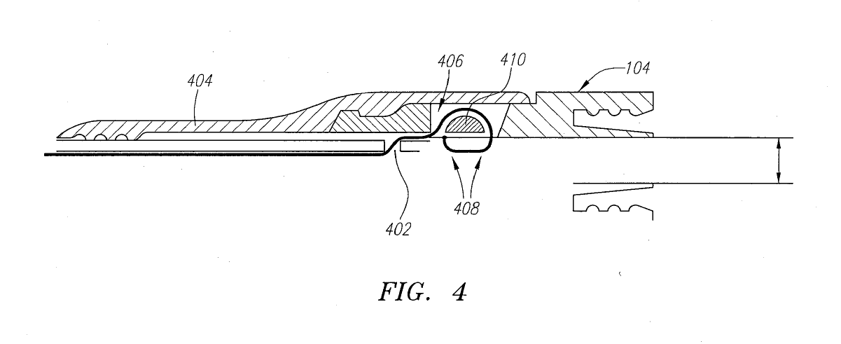

Referring to FIGS. 3a, 3b, and 3c, cross-sectional views depicting an exemplary embodiment of a catheter 100 with a “pig tail” loop 106 configuration as an anchoring mechanism, with the dynamic hub 104 in a completely disengaged position, partially disengaged position, and an engaged position, respectively, are shown. In FIG. 3a, the pigtail loop 106 portion of the distal end of the catheter 100 has been substantially straightened by releasing the tension on the cord 302. For purposes of this disclosure, one of ordinary skill in the art may appreciate that a cord, suture, thread or any other similar material may likewise be used to substantially perform the same objective. As herein depicted, the cord 302 is affixed at one end to the distal-most section of the catheter 100 and affixed at the other end to the dynamic hub 104—all the while, the cord 302 remains embedded within the catheter lumen of the tube member 110 except for its distal and proximal routing points. For example, at the distal end of the catheter 100, the cord 302 is routed out of a drilled suture hole 304 and routed back into the distal-most drilled suture hole 305. At the proximal end of the catheter 100, the cord 302 exits the catheter lumen through a suture hole 402 (See FIG. 4) and is routed around a routing lip 410, which is internal to the dynamic hub 104, and affixed to the outer circumference of the tube member 110 where it remains fixed. As shown, as the dynamic hub 104 is drawn longitudinally in a proximal direction, the cord 302 fold over itself internal to the dynamic hub 104, effectively traveling half the distance X necessary to form the pigtail loop 106 at the distal end. In an alternative embodiment, one of ordinary skill could use various termination points along the longitudinal axis of the tube member 110, including, for example, having the distal termination point occur back at the dynamic hub 104 or having a “closed loop” design whereby the cord 302 is manipulated to form one complete loop within the device itself.

The pigtail anchoring region 106 can have one or more series of perforations 304 that start and stop on different locations along the distal end of the catheter 100. The pitch, or the distance from one point on the perforation 304 to a corresponding point on an adjacent perforation 304 measured parallel to the axis of the tube member 110, may vary. For example, the perforations 304 may be spaced closer together at the proximal end, and farther apart at the distal end of the pigtail loop 106 portion, or vice versa. This configuration may facilitate the interfacing of the perforations 304 with different types of tissue encountered at various parts of the catheter. Additionally, the angles of the perforations 304 relative to the longitudinal axis of the tube member 110 may also vary. Moreover, the perforations 304 themselves may vary in cross-sectional geometry (i.e., semi-circular, triangular, trapezoid) and may be placed at one or more discrete locations along the tube member 110. For sake of clarity and simplicity, FIG. 3 depicts circular perforations configured normal to the catheter's 100 longitudinal axis.

The catheter 100 may be constructed of thermoplastic polymer such as polyurethane, ethyl vinyl acetate (EVA), polyether block amide elastomer, polypropylene, or polyolefin elastomers. The catheter system can also be constructed of a thermoset plastic like silicone. The pigtail anchoring region 106 may likewise be flexible but may be constructed of a different material than the remainder of the tube member 110.

Referring to FIG. 4, a cross-sectional view of the proximal end of a catheter 100, specifically focused on the internal structure of the dynamic hub 104, is shown. This enlarged detail partial-view of the dynamic hub 104 shows the strain relief seal 404, cavity 406, suture hole 402, and routed cord 302 around the routing lip 410. The strain relief 404 ensures that the proper seal will be maintained as the dynamic hub 104 translates along the tube member 110 and covers the suture holes to avoid possible leaks. As the cord 302 exits the tube member 110 through the suture hole 402, it is then routed up and over the routing lip 410, but nonetheless retains freedom of movement in the free open space of the cavity 406—said cavity 406 being otherwise completely sealed off. Once the cord 302 is funneled back towards the catheter lumen, it is suture preferred through the catheter lumen, thus, no corresponding suture hole is necessary. As the dynamic hub 104 is manipulated in a longitudinal direction, the internal circumference of the dynamic hub allows enough room between itself and the tube member 110 such that the cord 302 easily moves in between the dynamic hub 104 and the tube member 110.

The anchoring regions mentioned in the foregoing discussion and shown above in FIGS. 1 and 3c may replace or supplement the traditional anchoring mechanism embodied by a traditional pigtail loop shape or other shapes in the restraining portion of the catheter 100. Although not shown in the figures, the restraining portion of the catheter may vary as follows. The restraining portion as referenced herein may span one or more sections along the catheter that defines a traditional anchoring mechanism (embodied by the pigtail loop configuration or the malecot rib configuration). The length of the restraining portion may vary, according to the desired application. Typically, the restraining portion is located in the region medial to distal on the catheter, where the anchoring mechanism is to be activated in the body cavity. However, it is contemplated that the restraining portion can also be positioned closer to the proximal end of the catheter, as well as at multiple locations at any point between the proximal end and the distal end. In an exemplary embodiment, in addition to a first restraining portion comprising a pigtail configuration positioned near the distal end of the catheter, a second restraining portion comprising one or more anchoring geometries can be strategically positioned along the catheter between the proximal end and the first restraining portion, such that anchoring occurs at a tissue interface area in the body (e.g., at the skin of the patient).

Also contemplated herein are methods that can be performed using the subject devices or by other means. The methods can all comprise the act of providing a suitable device. Such provision can be performed by the end user. In other words, the “providing” merely requires the end user obtain, access, approach, position, set-up, activate, power-up or otherwise act to provide the requisite device in the subject method. Methods recited herein can be carried out in any order of the recited events which is logically possible, as well as in the recited order of events.

Exemplary embodiments, together with details regarding material selection and manufacture have been set forth above. As for other details of the presently described subject matter, these can be appreciated in connection with the above-referenced patents and publications as well as generally know or appreciated by those with skill in the art. The same can hold true with respect to method-based aspects in terms of additional acts as commonly or logically employed.

In addition, though the devices, systems and methods described herein have been presented herein in reference to exemplary embodiments, optionally incorporating various features, the devices, systems and methods described herein are not to be limited to that which is described or indicated as contemplated with respect to each variation. Various changes can be made to the subject matter described herein, and equivalents (whether recited herein or not included for the sake of some brevity) can be substituted without departing from the true spirit and scope of the disclosure.

Also, it is contemplated that any optional feature of the inventive variations described can be set forth and claimed independently, or in combination with any one or more of the features described herein. Stated otherwise, it is to be understood that each of the improvements described herein independently offer a valuable contributions to the state of the art. So too do the various other possible combination of the improvements/features described herein and/or incorporated by reference, any of which can be claimed.

Reference to a singular item, includes the possibility that there are plural of the same items present. More specifically, as used herein and in the appended claims, the singular forms “a,” “an,” “said,” and “the” include plural referents unless the specifically stated otherwise. In other words, use of the articles allow for “at least one” of the subject item in the description above as well as the claims below. It is further noted that the claims can be drafted to exclude any optional element. As such, this statement is intended to serve as antecedent basis for use of such exclusive terminology as “solely,” “only” and the like in connection with the recitation of claim elements, or use of a “negative” limitation.

Without the use of such exclusive terminology, the term “comprising” in the claims shall allow for the inclusion of any additional element—irrespective of whether a given number of elements are enumerated in the claim, or the addition of a feature could be regarded as transforming the nature of an element set forth in the claims. Likewise, use of the term “typically” does not exclude other possibilities. It can indicate a preference, however, for the stated characteristic. Except as specifically defined herein, all technical and scientific terms used herein are to be given as broad a commonly understood meaning as possible while maintaining claim validity.

1. A catheter comprising:
a longitudinal catheter shaft having a distal and a proximal end;
a cord in coaxial arrangement within said longitudinal catheter,
a static hub operatively connected to said longitudinal catheter at said proximal end,
a dynamic hub configured to move along a longitudinal axis of said longitudinal catheter substantially near said proximal end,
wherein a first end of said cord is affixed to said longitudinal catheter shaft at said proximal end and a second end of said cord is affixed to said longitudinal catheter shaft at said distal end, and further wherein the longitudinal movement of the dynamic hub is substantially one half the distance needed to close the pigtail shape.

2. The catheter of claim 1 wherein said movement of said dynamic hub in a proximal direction forms a pigtail curve at said distal end of said longitudinal catheter.

3. The catheter of claim 2, wherein said static hub extends circumferentially about said proximal end of said longitudinal catheter and has a first interlocking feature at an end adjacent to said dynamic hub.

4. The catheter of claim 3, wherein said dynamic hub extends circumferentially about said proximal end of said longitudinal catheter and has a second interlocking feature at an end adjacent to said static hub.

5. The catheter of claim 4 wherein said first and second interlocking features are configured to engage one another so as to temporarily join said dynamic hub and said static hub.

6. The catheter of claim 5 wherein, at said proximal end, said cord is routed out of said longitudinal catheter, around a cord engaging member, and affixed to said longitudinal catheter.

7. The catheter of claim 6 wherein, at said distal end, said cord is routed out of said longitudinal catheter and affixed to said longitudinal catheter at a still further distal point.

8. The catheter of claim 7, wherein said longitudinal catheter shaft has a plurality of perforations at said distal end.

9. The catheter of claim 7, wherein said catheter and hubs are constructed of thermoplastic polymers.

10. The catheter of claim 8, wherein said catheter is inserted into a body cavity.

11. A drainage catheter comprising:
a catheter means having a distal and a proximal end;
a cord means in coaxial arrangement within said catheter,
a first hub means operatively connected to said catheter means at said proximal end,
a second hub means configured to move along a longitudinal axis of said catheter means, and
wherein a first end of said cord means is affixed to said catheter means at said proximal end and a second end of said cord means is affixed to said catheter means at said distal end, and further wherein longitudinal movement of said second hub is substantially one half the distance needed to close the pigtail shape.

12. The catheter of claim 10 wherein said movement of said first hub means in a proximal direction forms a pigtail curve at said distal end of said catheter means.

13. The catheter of claim 11, wherein said second hub means extends circumferentially about said proximal end of said catheter means and has a first interlock means at an end adjacent to said first hub means.

14. The catheter of claim 12, wherein said first hub means extends circumferentially about said proximal end of said catheter means and has a second interlock means at an end adjacent to said second hub means.

15. The catheter of claim 13, wherein said first and second interlock means are configured to engage one another.

16. The catheter of claim 14 wherein, at said proximal end, said cord means is routed to an external portion of said catheter means, around a cord engaging member means, and affixed to said catheter means.

17. The catheter of claim 15 wherein, at said distal end, said cord means is routed to an external portion of said catheter means and affixed to said catheter means at a still further distal point.

18. The catheter of claim 16, wherein said catheter means and said hub means are constructed of thermoplastic polymers.

19. The catheter of claim 17, wherein said catheter means is inserted into a body cavity.

20. A method of fixing a catheter within a body cavity comprising:
inserting a distal end of a catheter into a body cavity, the catheter comprising a longitudinal catheter shaft having a distal and a proximal end, a cord in coaxial arrangement within said longitudinal catheter, a static hub operatively connected to said longitudinal catheter at said proximal end, a dynamic hub configured to move along a longitudinal axis of said longitudinal catheter substantially near said proximal end and resting in a disengaged state wherein a first end of said cord is affixed to said dynamic hub and a second end of said cord is affixed to said longitudinal catheter at said distal end; and
securing the catheter within the body cavity by engaging said dynamic hub to said static hub and causing the distal portion said longitudinal catheter to form a pigtail curve.
